(19) United States
(12) Patent Application Publication
(10) Pub. No.: US 2024/0215954 A1
(43) Pub. Date: Jul. 4, 2024

(54) ULTRASOUND IMAGING SYSTEM AND METHOD FOR CALCULATING AND DISPLAYING A PROBE POSITION ADJUSTMENT

(71) Applicant: GE Precision Healthcare LLC, Wauwatosa, WI (US)

(72) Inventors: Krishna Seetharam Shriram, Bangalore (IN); Chandan Kumar Mallappa Aladahalli, Bangalore (IN); Christian Perrey, Mondsee (AT); Michaela Hofbauer, Linz (AT)

(21) Appl. No.: 18/145,631

(22) Filed: Dec. 22, 2022

(51) Int. Cl. A61B 8/08; A61B 8/00

(52) U.S. Cl. CPC A61B 8/483; A61B 8/54

(57) ABSTRACT

An ultrasound imaging system and method for calculating and displaying a probe position adjustment. The method includes acquiring a volumetric dataset with an ultrasound probe in a volumetric acquisition mode. The method includes automatically identifying, with a processor, an object representing a structure-of-interest from the volumetric dataset. The method includes automatically identifying, with the processor, an axis of the structure-of-interest based on the object. The method includes automatically calculating, with the processor, a probe position adjustment from a current probe position to enable the acquisition of a target scan plane of the structure-of-interest that either includes and is parallel to the axis or is perpendicular to the axis. The method includes presenting the probe position adjustment on a display device.

### FIELD OF THE INVENTION

This disclosure relates generally to an ultrasound imaging system and method for using a volumetric ultrasound dataset to calculate a display a probe position adjustment with respect to an axis of a structure-of-interest.

### BACKGROUND OF THE INVENTION

Ultrasound imaging is an imaging modality that uses ultrasonic signals (i.e., sound waves) to produce images of a patient's anatomy. Ultrasound imaging has become a commonly used imaging modality for a number of reasons. For instance, ultrasound imaging is relatively low-cost compared to many other imaging modalities, ultrasound imaging does not rely on ionizing radiation to generate images, and ultrasound imaging may be performed as a real-time imaging modality. For these and other reasons, ultrasound imaging is commonly used to image and analyze various structures-of-interest within a patient's body in order to evaluate the patient's condition and/or determine a medical diagnosis.

Conventional ultrasound imaging systems are used to evaluate a structure-of-interest according to many ultrasound protocols. It is oftentimes desired to obtain a measurement related to the structure-of-interest in order to evaluate the patient's condition. For example, when evaluating ovarian masses in a patient, the clinician acquires ultrasound images from the adnexa. It is desired to quantitatively evaluate the sizes of any ovarian masses in order to accurately evaluate and/or diagnose the patient.

Conventional ultrasound imaging systems have anisotropic resolution. The resolution is typically better in insonated scan planes compared to planes that cross one or more insonated scan planes and are reconstructed from volumetric data. An A-plane is a common example of an insonated scan plane. Conventional two-dimensional images are examples of images representing directly insonated scan planes. In other words, the two-dimensional image represents the insonated scan plane. A C-plane and an oblique plane are both examples of planes reconstructed from volumetric data that cross one or more insonated scan planes. An image representing a C-plane or an image representing an oblique plane may be generated by performing a multiplanar reconstruction (MPR) based on the volumetric ultrasound data.

It is well-known that the resolution and image quality of images generated by a multiplanar reconstruction (MPR) are inferior to the resolution and image quality of images representing directly insonated scan planes. For this reason, when taking a measurement of a structure-of-interest, it is typically desirable to have the axis along which the measurement is desired to be included in the insonated scan plane. According to conventional techniques, a user may enter a two-dimensional imaging mode and attempt to position the ultrasound probe to include the desired axis within the scan plane. This is challenging and time-consuming for clinicians. It can be extremely difficult to determine if the ultrasound probe is positioned properly to image and measure an axis of a structure-of-interest while in a two-dimensional imaging mode.

For at least these reasons, there is a need for an improved method and ultrasound imaging system for calculating and displaying a probe position adjustment with respect to a current probe position of the ultrasound probe.

### BRIEF DESCRIPTION OF THE INVENTION

The above-mentioned shortcomings, disadvantages and problems are addressed herein which will be understood by reading and understanding the following specification.

In an embodiment, a method of ultrasound imaging includes acquiring a volumetric dataset with an ultrasound probe in a volumetric acquisition mode. The method includes automatically identifying, with a processor, an object representing a structure-of-interest based on the object. The method includes automatically identifying, with the processor, an axis of the structure-of-interest based on the object. The method includes automatically calculating, with the processor, a probe position adjustment from a current probe position to enable the acquisition of a target scan plane of the structure-of-interest that either includes and is parallel to the axis or is perpendicular to the axis. The method includes presenting the probe position adjustment on a display device.

In an embodiment, an ultrasound imaging system includes an ultrasound probe, a display device, and a processor in electronic communication with both the ultrasound probe and the display device. The processor is configured to control the ultrasound probe to acquire a volumetric dataset in a volumetric acquisition mode. The processor is configured to automatically identify an object from the volumetric dataset representing a structure-of-interest. The processor is configured to automatically identify an axis of the structure-of-interest based on the object. The processor is configured to automatically calculate a probe position adjustment from a current probe position to enable the acquisition of a target scan plane of the structure-of-interest that either includes and is parallel to the axis or is perpendicular to the axis. The processor is configured to present the probe position adjustment on the display device.

Various other features, objects, and advantages of the invention will be made apparent to those skilled in the art from the accompanying drawings and detailed description thereof.

### BRIEF DESCRIPTION OF THE DRAWINGS

FIG. 1 is a schematic diagram of an ultrasound imaging system in accordance with an embodiment;

FIG. 2 is a flow chart of a method in accordance with an embodiment;

FIG. 3 is a flow chart of a method in accordance with an embodiment;

FIG. 4 is a representation of a translational scan path used to acquire a volumetric dataset in accordance with an embodiment;

FIG. 5 is a representation of an acquisition used to acquire a volumetric dataset in accordance with an embodiment;

FIG. 6 is representation of an oblique plane shown with respect to both an ultrasound probe and a plurality of scan planes in accordance with an embodiment;

FIG. 7 is a representation of the structure-of-interest in accordance with an embodiment;

FIG. 8 is a representation of the structure-of-interest with respect to a scan plane in accordance with an embodiment;

FIG. 9 is a representation of an ultrasound probe with respect to three axes and a scanned volume in accordance with an exemplary embodiment;

FIG. 10 is a representation of a graphical display in accordance with an exemplary embodiment;

FIG. 11 is a representation of four frames of a video loop that may be displayed in a sequence or repeating loop to convey the probe position adjustment in accordance with an exemplary embodiment; and

FIG. 12 is a representation of a two-dimensional image 990 in accordance with an exemplary embodiment.

### DETAILED DESCRIPTION OF THE INVENTION

In the following detailed description, reference is made to the accompanying drawings that form a part hereof, and in which is shown by way of illustration specific embodiments that may be practiced. These embodiments are described in sufficient detail to enable those skilled in the art to practice the embodiments, and it is to be understood that other embodiments may be utilized, and that logical, mechanical, electrical and other changes may be made without departing from the scope of the embodiments. The following detailed description is, therefore, not to be taken as limiting the scope of the invention.

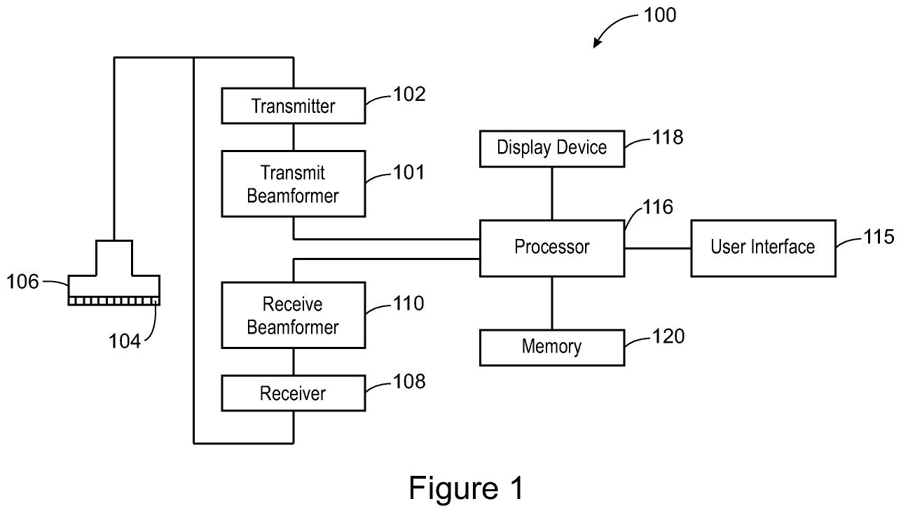

FIG. 1 is a schematic diagram of an ultrasound imaging system 100 in accordance with an embodiment. The ultrasound imaging system 100 includes a transmit beamformer 101 and a transmitter 102 that drive elements 104 within an ultrasound probe 106 to emit pulsed ultrasonic signals into a body (not shown) through one or more transmit events. The ultrasound probe 106 may be any type of ultrasound probe capable of a three-dimensional (3D) or a four-dimensional (4D) acquisition. For example, the ultrasound probe 106 may be a 2D matrix array probe, a mechanical 3D/4D probe, or any other type of ultrasound probe configured to acquire volumetric ultrasound data. According to other embodiments the ultrasound probe 106 may be configured to acquire volumetric ultrasound data by being translated across the patient while acquiring a sequence of two-dimensional images. Still referring to FIG. 1, the pulsed ultrasonic signals are back-scattered from structures in the body, like blood cells or muscular tissue, to produce echoes that return to the elements 104. The echoes are converted into electrical signals by the elements 104 and the electrical signals are received by a receiver 108. The electrical signals representing the received echoes are passed through a receive beamformer 110 that outputs ultrasound data. According to some embodiments, the probe 106 may contain electronic circuitry to do all or part of the transmit beamforming and/or the receive beamforming. For example, all or part of the transmit beamformer 101, the transmitter 102, the receiver 108 and the receive beamformer 110 may be situated within the ultrasound probe 106. The terms “scan” or “scanning” may also be used in this disclosure to refer to acquiring data through the process of transmitting and receiving ultrasonic signals. The terms “data” and “ultrasound data” may be used in this disclosure to refer to either one or more datasets acquired with an ultrasound imaging system. A user interface 115 may be used to control operation of the ultrasound imaging system 100. The user interface 115 may be used to control the input of patient data, or to select various modes, operations, parameters, and the like. The user interface 115 may include one or more user input devices such as a keyboard, hard keys, a touch pad, a touch screen, a track ball, rotary controls, sliders, soft keys, or any other user input devices.

The ultrasound imaging system 100 also includes a processor 116 to control the transmit beamformer 101, the transmitter 102, the receiver 108 and the receive beamformer 110. The user interface 115 is in electronic communication with the processor 116. The processor 116 may include one or more central processing units (CPUs), one or more microprocessors, one or more microcontrollers, one or more graphics processing units (GPUs), one or more digital signal processors (DSPs), and the like. According to some embodiments, the processor 116 may include one or more GPUs, where some or all of the one or more GPUs include a tensor processing unit (TPU). According to embodiments, the processor 116 may include a field-programmable gate array (FPGA), or any other type of hardware capable of carrying out processing functions. The processor 116 may be an integrated component or it may be distributed across various locations. For example, according to an embodiment, processing functions associated with the processor 116 may be split between two or more processors based on the type of operation. For example, embodiments may include a first processor configured to perform a first set of operations and a second, separate processor to perform a second set of operations. According to embodiments, one of the first processor and the second processor may be configured to implement a neural network. The processor 116 may be configured to execute instructions accessed from a memory. According to an embodiment, the processor 116 is in electronic communication with the ultrasound probe 106, the receiver 108, the receive beamformer 110, the transmit beamformer 101, and the transmitter 102. For purposes of this disclosure, the term “electronic communication” may be defined to include both wired and wireless connections. The processor 116 may control the ultrasound probe 106 to acquire ultrasound data. The processor 116 controls which of the elements 104 are active and the shape of a beam emitted from the ultrasound probe 106. The processor 116 is also in electronic communication with a display device 118, and the processor 116 may process the ultrasound data into images for display on the display device 118. According to embodiments, the processor 116 may also include a complex demodulator (not shown) that demodulates the RF data and generates raw data. In another embodiment, the demodulation may be carried out earlier in the processing chain. The processor 116 may be adapted to perform one or more processing operations according to a plurality of selectable ultrasound modalities on the data. The data may be processed in real-time during a scanning session as the echo signals are received. The processor 116 may be configured to scan-convert the ultrasound data acquired with the ultrasound probe 106 so it may be displayed on the display device 118. Displaying ultrasound data in real-time may involve displaying images based on the ultrasound data without any intentional delay. For example, the processor 116 may display each updated image frame as soon as each updated image frame of ultrasound data has been acquired and processed for display during the process of an ultrasound procedure. Real-time frame rates may vary based on the size of the region or volume from which data is acquired and the specific parameters used during the acquisition. According to other embodiments, the data may be stored temporarily in a buffer (not shown) during a scanning session and processed in less than real-time. According to embodiments that include a software beamformer, the functions associated with the transmit beamformer 101 and/or the receive beamformer 108 may be performed by the processor 116.

According to various embodiments, the components illustrated in FIG. 1 may be part of a distributed ultrasound imaging system. For example, one or more of the processor 116, the user interface 115, the transmitter 102, the transmit beamformer 101, the receive beamformer 110, the receiver 108, a memory 120, and the display device 118 may be located remotely from the ultrasound probe 106. The aforementioned components may be located in different rooms or different facilities according to various embodiments. For example, the probe 106 may be used to acquire ultrasound data from the patient and then transmit the ultrasound data, via either wired or wireless techniques, to the processor 116.

According to an embodiment, the ultrasound imaging system 100 may continuously acquire ultrasound data at a volume rate of, for example, 10 Hz to 30 Hz. Images generated from the data may be refreshed at similar frame-rates. Other embodiments may acquire data and display images at different rates. For example, some embodiments may acquire ultrasound data at a volume rate of less than 10 Hz or greater than 30 Hz depending on the size of each frame of data and the parameters associated with the specific application. The memory 120 is included for storing processed frames of acquired data. In an exemplary embodiment, the memory 120 is of sufficient capacity to store frames of ultrasound data acquired over a period of time at least several seconds in length. The frames of data are stored in a manner to facilitate retrieval thereof according to its order or time of acquisition. The memory 120 may comprise any known data storage medium.

In various embodiments of the present invention, data may be processed by other or different mode-related modules by the processor 116 (e.g., B-mode, color flow Doppler, M-mode, color M-mode, spectral Doppler, Elastography, TVI, strain, strain rate, and the like) to form two-dimensional ultrasound data or three-dimensional ultrasound data. For example, one or more modules may generate B-mode, color Doppler, M-mode, color M-mode, spectral Doppler, Elastography, TVI, strain, strain rate and combinations thereof, and the like. The image beams and/or frames are stored, and timing information indicating a time at which the data was acquired in memory may be recorded. The modules may include, for example, a scan conversion module to perform scan conversion operations to convert the image frames from beam space coordinates to display space coordinates. A video processor module may be provided that reads the image frames from a memory, such as the memory 120, and displays the image frames in real-time while a procedure is being carried out on a patient. The video processor module may store the image frames in an image memory, from which the images are read and displayed.

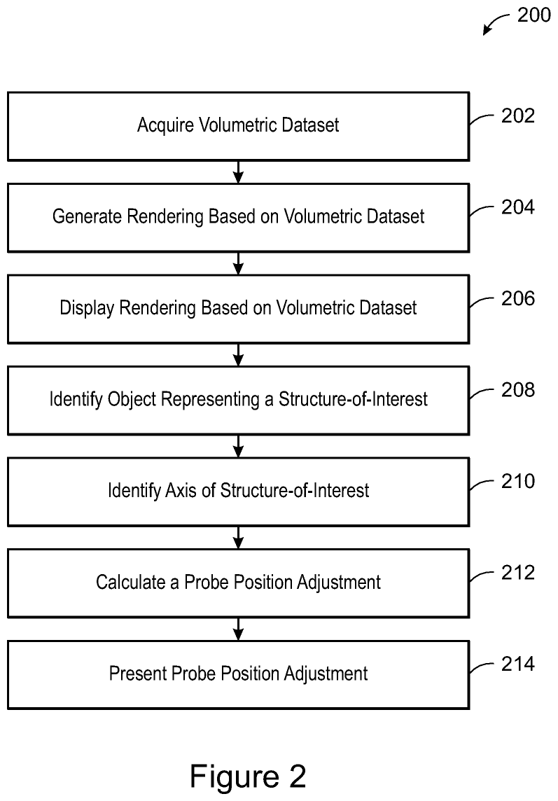

FIG. 2 is a flow chart of a method 200 in accordance with an exemplary embodiment. The individual blocks of the flow chart represent steps that may be performed in accordance with the method 200. Additional embodiments may perform the steps shown in a different sequence and/or additional embodiments may include additional steps not shown in FIG. 2. The technical effect of the method 200 is the calculation and display of a probe position adjustment with respect to a current probe position of the ultrasound probe 106. The method 200 will be described according to an embodiment where it is performed with the ultrasound imaging system 100 shown in FIG. 1. However, it should be appreciated by those skilled in the art that the method 200 may be performed with other ultrasound imaging systems according to various embodiments. The method 200 will be described in detail hereinafter.

At step 202, the processor 116 controls the ultrasound probe 106 to acquire a volumetric dataset. The processor 116 may control the ultrasound probe 106 to acquire the volumetric dataset according to a variety of different techniques. As discussed previously, the ultrasound probe 106 may be a 2D matrix array probe with full beam-steering in both an azimuth and an elevation direction. For embodiments where the ultrasound probe 106 is a 2D matrix array, the processor 116 may be configured to control the ultrasound probe 106 to acquire the volumetric dataset by acquiring data from a plurality of separate scan planes at different angles as is known by those skilled in the art. The ultrasound probe 106 may be a mechanically rotating probe including an array of elements that is mechanically swept or rotated in order to acquire information from scan planes disposed at a plurality of different angles as is known by those skilled in the art. The ultrasound probe may also be a one-dimensional (1D) array probe, that is configured to be translated across the patient to acquire the volumetric dataset. For embodiments that involve translating a 1D array probe, the ultrasound imaging system 100 may additionally include a position sensing system to identify the relative positions of the ultrasound probe, and therefore the scan plane, at each respective position while the ultrasound probe 106 is translated. According to other embodiments, the processor 116 may be configured to use image processing techniques and/or artificial intelligence techniques in order to determine the relative positions of the various scan planes acquired while translating the ultrasound probe 106. For purposes of this disclosure, the term “volumetric dataset” will be defined to include one or more volumes of ultrasound data. For embodiments, where the volumetric dataset includes more than one volume of ultrasound data, each volume of ultrasound data may have been acquired at a different time. The method 200 will be described according to an exemplary embodiment where the volumetric dataset is a single volume of ultrasound data.

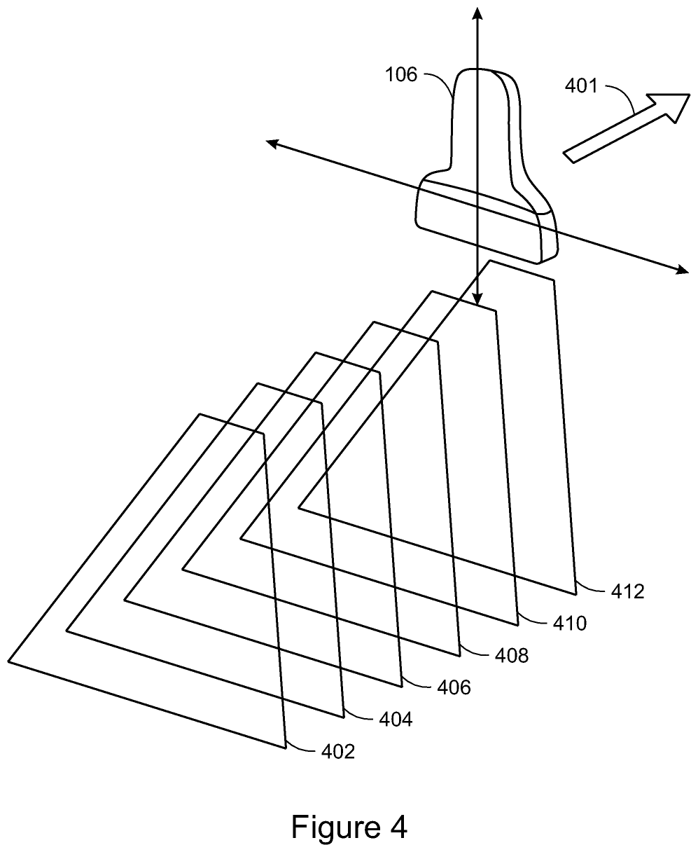

FIG. 4 is a representation of a translational scan path used to acquire a volumetric dataset according to an exemplary embodiment. FIG. 4 will be used to show how the ultrasound probe 106 may be translated in order to acquire the volumetric dataset. According to an embodiment, the ultrasound probe 106 acquires a two-dimensional image from a plurality of different locations while the ultrasound probe 106 is translated in a direction as indicated by an arrow 401. For example, FIG. 4 includes a first scan plane 402, a second scan plane 404, a third scan plane 406, a fourth scan plane 408, a fifth scan plane 410, and a sixth scan plane 412. FIG. 4 includes representations of six scan planes, but it should be appreciated by those skilled in the art that other embodiments may acquired information from more than six separate scan planes. The processor 116 combines the information acquired from each of the scan planes into a volumetric dataset according to an exemplary embodiment. As discussed hereinabove, the processor 116 may use either information from a position sensor attached to the ultrasound probe 106 and/or information from the images acquired from each of the scan planes to register the scan planes to each other in order to generate the volumetric dataset. For example, the processor 116 may use imaging processing techniques and/or artificial intelligence techniques to combine the information from each of the scan planes into the volumetric dataset.

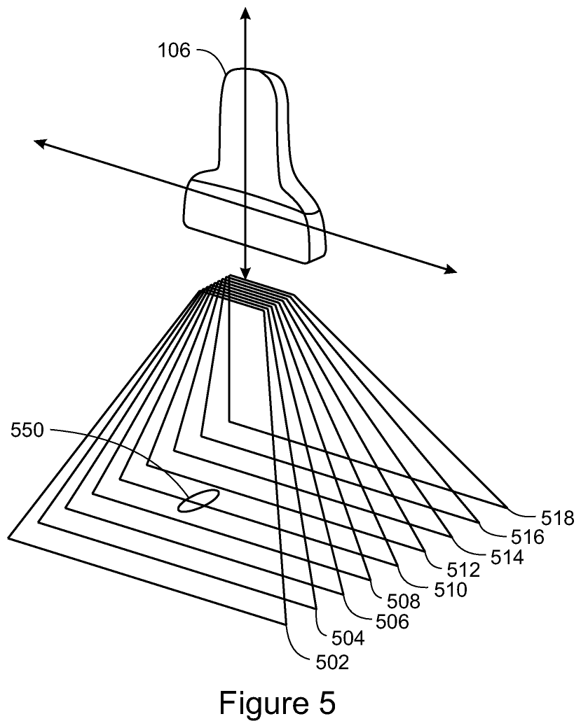

FIG. 5 is a representation of an acquisition used to acquire a volumetric dataset according to an exemplary embodiment. FIG. 5 includes the ultrasound probe 106, and a plurality of scan planes shown in spatial relationship with respect to the ultrasound probe 106. FIG. 5 includes representations of nine scan planes for illustrative purposes. It should be appreciated by those skilled in the art that embodiments may include either more nine scan planes or fewer than nine scan planes. For most embodiments, it is anticipated that more than nine scan planes will be used. FIG. 5 includes a first scan plane 502, a second scan plane 504, a third scan plane 506, a fourth scan plane 508, a fifth scan plane 510, a sixth scan plane 512, a seventh scan plane 514, an eighth scan plane 516, and a ninth scan plane 518. Each of the scan planes represented in FIG. 5 are shown at a different angle with respect to the ultrasound probe 106. Unlike the embodiment shown in FIG. 4, in the embodiment shown in FIG. 5, the ultrasound probe 106 is not translated during the acquisition of the volumetric dataset. According to an embodiment where the ultrasound probe 106 is a mechanically rotating ultrasound probe, the ultrasound probe 106 may include a transducer array that is mechanically rotated to enable the acquisition of ultrasound data from a plurality of scan planes at different rotational positions with respect to a body of the ultrasound probe 106. The ultrasound probe 106 may be configured to continuously sweep the transducer array back-and-forth to acquire a plurality of volumetric datasets as is known by those skilled in the art. It should be appreciated that according to an embodiment where the transducer array is configured to sweep back- and forth, the ultrasound probe 106 may be configured to alternate the order in which ultrasound data from the scan planes is acquired. For example, a first volumetric dataset may be acquired by acquiring the first scan plane 502, the second scan plane 504, the third scan plane 506, the fourth scan plane 508, the fifth scan plane 510, the sixth scan plane 512, the seventh scan plane 514, the eighth scan plane 516, and the ninth scan plane 518 in that order. However, the next volumetric dataset may be acquired by acquiring the ninth scan plane 518, the eighth scan plane 516, the seventh scan plane 514, the sixth scan plane 512, the fifth scan plane 510, the fourth scan plane 508, the third scan plane 506, the second scan plane 504 and then the first scan plane 502 in that order.

Each of the scan planes shown in FIG. 4 and FIG. 5 represent a insonated scan plane—in other words, with the ultrasound probe 106 positioned as shown in FIG. 4 and FIG. 5 respectively, the processor 106 may be configured to display a two-dimensional image from any of the scan planes represented in the respective figure without applying a multiplanar reconstruction to the volumetric dataset. This means that resolution and image quality of images representing the illustrated scan planes will have improved resolution and image quality compared to an image generated by applying a multiplanar reconstruction to the volumetric dataset, such as an image of a C-plane or an image of an oblique plane.

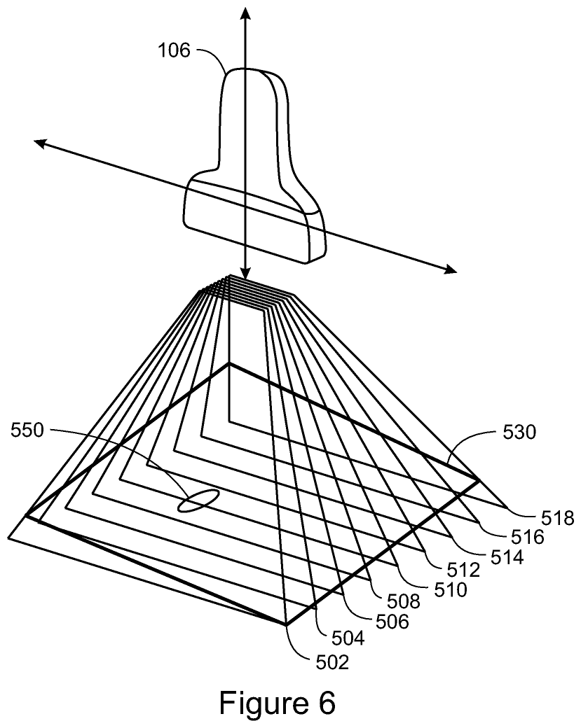

FIG. 6 is representation of an oblique plane 530 shown with respect to both the ultrasound probe 106 and the scan planes previously shown in FIG. 5 in accordance with an embodiment. As is clear based on FIG. 6, the oblique plane cuts across two or more of the insonated scan planes (i.e., the first scan plane 502, the second scan plane 504, the third scan plane 506, the fourth scan plane 508, the fifth scan plane 510, the sixth scan plane 512, the seventh scan plane 514, the eighth scan plane 516, and the ninth scan plane 518). As such, those skilled in the art will appreciate that, in order to visualize the oblique plane 530, it is necessary to perform a multiplanar reformat (MPR) based on the volumetric dataset. It is clearly not possible to visualize all of the oblique plane 530 based on just the ultrasound data acquired along any one of the insonated scan planes illustrated in FIG. 6. Furthermore, it is not physically possible to acquire ultrasound data by only insonating the oblique plane due to physical limitations of ultrasound imaging.

Referring back to FIG. 2, at the step 204, the processor 116 generates a rendering based on the volumetric dataset. The rendering may be, for instance: a volume-rendered image, such as a volume rendering; a projection image such as a maximum intensity projection (MIP) image, a minimum intensity projection (MinIP) image; or multiplanar reformat (MPR) image; or any other type of rendering generated based on the volumetric dataset acquired at step 202.

At step 206, the processor 116 displays the rendering generated at step 204 on the display device. Both steps 204 and 206 are optional. Some embodiments may include steps 204 and 206, while steps 204 and 206 may be omitted according to other embodiments. For embodiments where steps 204 and 206 are omitted, the method 200 may proceed directly from step 202 to step 208.

At step 208, the processor 116 identifies an object representing a structure-of-interest. A structure-of-interest 550 is shown with respect to FIG. 5 and FIG. 6. The structure-of-interest 550 may be an ovarian mass (also commonly referred to as an ovarian cyst) according to an exemplary embodiment. The processor 116 may be configured to identify the object representing the structure-of-interest 550 directly from the volumetric dataset or the processor 116 may be configured to identify the object representing the structure-of-interest from one or more renderings generated from the volumetric dataset.

According to an embodiment, the processor 116 may be configured to identify the object representing the structure-of-interest 550 from the volumetric dataset using artificial intelligence techniques. For example, the processor 116 may be configured to implement a trained artificial intelligence technique, such as a trained neural network, to identify the object representing the structure-of-interest 550 from the volumetric dataset. The neural network may be a convolutional neural network (CNN) according to an exemplary embodiment. The neural network may be a U-net according to various embodiments. It should be appreciated by those skilled in the art that other types of neural networks may be used according to various embodiments.

According to an embodiment, the processor 116 may be configured to identify the object representing the structure-of-interest 550 from the volumetric dataset using image processing techniques. For example, the processor 116 may be configured to use one or more image processing techniques to identify the object representing the structure-of-interest 550 from the volumetric dataset. A non-limiting list of image processing techniques that may be used by the processor 116 to identify the object representing the structure-of-interest 550 includes thresholding techniques, connected component analyses, and shape-based identification techniques. It should be appreciated by those skilled in the art that other types of image processing techniques may be used according to various embodiments.

By identifying the object representing the structure-of-interest 550 from the volumetric dataset, the processor 116 is able to search for the object representing the structure-of-interest 550 in the entire volume instead of just a single two-dimensional image as is standard with conventional techniques. This is particularly advantageous for situations where the object representing the structure-of-interest 550 is not positioned within any of the scan planes.

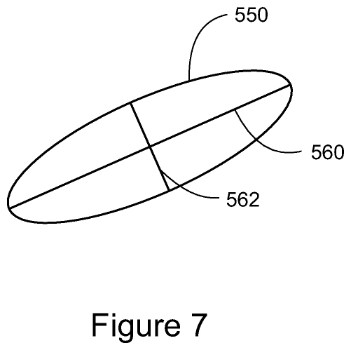

FIG. 7 is a representation of the structure-of-interest 550 according to an exemplary embodiment. The structure-of-interest 550 represented in FIG. 7 is ellipsoidal in shape. Ovarian masses are oftentimes generally ellipsoidal in shape. According to an exemplary embodiment, the structure-of-interest 550 may be an ovarian mass. However, in other embodiments, the structure-of-interest 550 may be an anatomical structure other than an ovarian mass. FIG. 7 is a two-dimensional representation of a three-dimensional shape. As such, the structure-of-interest 550 is represented as an ellipse in FIG. 7. Those skilled in the art should appreciate that the structure-of-interest 550 extends in an out-of-plane direction that is not represented in FIG. 7.

A long-axis 560 and a short axis 562 are represented on the structure-of-interest 550. As discussed hereinabove, most ovarian masses are generally ellipsoidal in shape. As such, a two-dimensional image including an ovarian mass will typically be generally elliptical in shape. For embodiments where the structure-of-interest is generally ellipsoidal, the long-axis 560 may correspond to a major axis of the ellipse and the short axis 562 may correspond to a minor axis of the ellipse. In the embodiment shown in FIG. 7, the structure-of-interest 550 is generally ellipsoidal, and therefore the long axis 560 corresponds to the major axis of the structure-of-interest 550 and the short axis 562 corresponds to the minor axis of the structure-of-interest 550.

The processor 116 may be configured to identify the long axis 560 by identifying the position and orientation of a straight line with the maximum length within the structure-of-interest 550. The processor 116 may be configured to identify the long axis 560 using artificial intelligence techniques or image processing techniques. Examples of artificial intelligence techniques that may be used include implementing a trained neural network, such as a deep neural network, a convolutional neural network (CNN). According to some embodiments, the CNN may be a U-Net or any other type of convolutional neural network.

Embodiments may implement one or more image processing techniques to identify the straight line with the maximum length within the structure-of-interest 550. For example, according to an exemplary embodiment, the processor 116 may be configured to first identify a boundary of the object. Volumetric datasets are oftentimes described in terms of a plurality of volume elements called voxels. The processor 116 may, for instance, identify all of the voxels associated with the boundary of the object. The processor 116 may then calculate a distance from each voxel located on the boundary to each of the other voxels that represent the boundary of the object. Next, the processor 116 may be configured to identify the longest distance between two of the voxels associated with the boundary of the object. The longest distance between two of the boundary voxels may be considered to be the long axis according to some embodiments.

According to another embodiment, the processor 116 may be configured to determine a center of gravity for the object. The center of gravity is a point or location within the object that represents the balance point for the object. The processor 116 may assign the same weight to every voxel in the object when calculating the center of gravity. For example, in the case of a system of voxels Vᵢ=1, . . . , n, each with mass mi that are located in space with coordinates rᵢ, 1, . . . , n, the coordinates R of the center of mass satisfy the condition show below in equation 1:

$${\sum }_{i = 1}^{n} {m}_{i} ( {r}_{i} - R ) = 0 \qquad ( 1 )$$

Therefore, the coordinates R of the center of mass may be found by solving the equation 1 for R, which results in equation 2, where M is the total mass of all the voxels:

$$R = \frac{1}{M} {\sum }_{i = 1}^{n} {m}_{i} {r}_{i} \qquad ( 2 )$$

It should be appreciated by those skilled in the art that the processor 116 may be configured to calculate the center of mass using one or more different techniques according to various embodiments.

According to an embodiment, processor 116 may identify the long axis by identifying the longest line passing through the center of gravity that connects two boundary voxels of the object. That is, the long axis may be defined as the longest straight line between two boundary voxels that passes through the center of gravity of the object according to various embodiments.

According to various embodiments the processor may be configured to identify the short axis of the object at step 210. For example, the processor 116 may be configured to use the position of the center of gravity of the object to identify a short axis of the object. The short axis may, for instance, be defined to be the shortest straight line connecting two voxels on the boundary of the object that passes through the center of gravity. According to some embodiments, the short axis may be defined to be perpendicular to a long axis of the object. It should be appreciated by those skilled in the art that one or both of the long axis and the short axis may be defined and/or calculated differently according to various embodiments.

According to another embodiment, the processor 116 may be configured to identify, based on the volumetric dataset, a plane through the object where the object has a maximum plane area. In other words, the processor 116 may be configured to identify the position of a plane intersecting the object that maximizes the plane area of the object on the plane. For example, the processor 116 may be configured to iteratively calculate a plane area of the object for a plurality of different plane orientations until a plane with a maximum plane area has been identified. For shapes that are generally ellipsoidal in shape, the plane that maximizes the plane area of the object will coincide with the a long-axis of the ellipsoid.

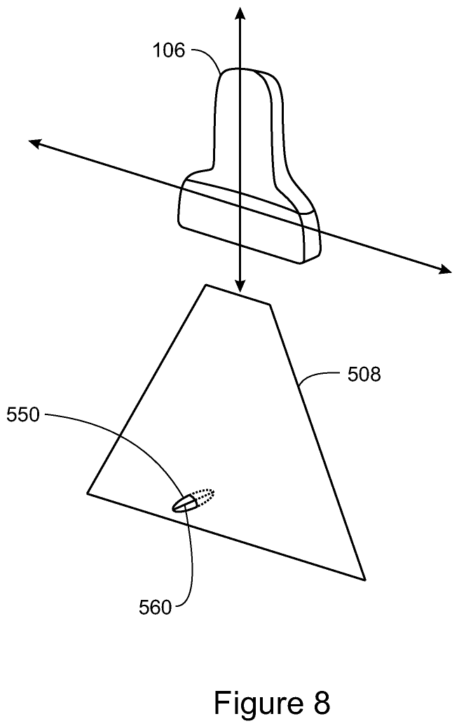

FIG. 8 is a representation of the structure-of-interest 550 with respect to the fourth scan plane 508. The fourth scan plane 508 is in the same position with respect to the ultrasound probe 106 in both FIG. 8 and FIG. 5. FIG. 8 clearly illustrates how the long axis 560 of the structure-of-interest 550 is not included in the fourth scan plane 508. In FIG. 8, the structure-of-interest 550 and the long axis 560 are shown in both solid line and dashed line. In FIG. 8, the portion of the structure-of-interest 550 and the major axis 560 in front of the fourth scan plane 508 are shown in solid line, and the portion of the structure-of-interest 550 and the long axis 560 behind the fourth scan plane 508 are shown in dashed line. FIG. 8 further helps to illustrate how the structure-of-interest 550 is ellipsoidal according to an embodiment. Based on the illustration shown in FIG. 8, it is easy to see that the fourth scan plane 508 does not include the major axis 560. Furthermore, none of the scan planes illustrated in FIG. 5 or FIG. 6 include the major axis 560 either.

Referring back to FIG. 2, at step 212, the processor 116 calculates a probe position adjustment. The probe position adjustment is an adjustment that needs to be applied to a current probe position of the ultrasound probe 106 in order to position the ultrasound probe 106 in a position and orientation to acquire two-dimensional ultrasound data from a scan plane that either includes an axis of the structure-of-interest 550 or is perpendicular to the axis of the structure-of-interest 550. The method 200 will be described according to an exemplary embodiment where is it desired to include the axis of the structure-of-interest in an insonated scan plane.

The position of the ultrasound probe 106 with respect to the structure-of-interest 550 is known by the processor 116 based on the position of the object identified in the volumetric ultrasound dataset. Based on this known relationship between the ultrasound probe 106 and the structure-of-interest, it is possible for the processor 116 to calculate the probe position adjustment that needs to be applied to the current probe position in order to acquire two-dimensional ultrasound data from a scan plane that either includes the axis or is perpendicular to the axis. For example, the processor 116 may first identify the position of the scan plane that either includes the axis or is perpendicular to the axis, and then, based on the position of the scan plane, the processor calculates the probe position adjustment that needs to be applied to the ultrasound probe 106 to position the ultrasound probe into a position where it is possible to acquire the desired scan plane by directly isonating the desired scan plane. For example, according to an embodiment, the processor 116 may be configured to calculate the probe position adjustment that would need to be applied to the current probe position to acquire two-dimensional ultrasound data from a scan plane that includes the long axis 560. According to another embodiment the processor 116 may be configured to calculate the probe position adjustment that would need to be applied to the current probe position to acquire two-dimensional ultrasound data from a scan plane that is perpendicular to the long axis 560. A scan plane that includes the short axis 562 is one example of a scan plane that is perpendicular to the long axis 560. According to another embodiment the processor 116 may be configured to calculate the probe position adjustment that would need to be applied to the current probe position to acquire two-dimensional ultrasound data from a scan plane that includes the short axis 562. According to another embodiment the processor 116 may be configured to calculate the probe position adjustment that would need to be applied to the current probe position to acquire two-dimensional ultrasound data from a scan plane that is perpendicular to the short axis 562.

As discussed previously, generating a two-dimensional image by insonating the desired scan plane advantageously provides an image with better resolution and image quality than is available by generating an image using multiplanar reformat from a volumetric dataset. A two-dimensional image is, by definition, acquired by insonating the scan plane represented by the image. As such, it is always desirable to use a two-dimensional image over an image generated using a multiplanar reformat (MPR) from volumetric data for determining measurements. Taking measurement from an image acquired in a two-dimensional imaging mode is therefore currently the best practice for sonographers.

Next, at step 214, the processor presents the probe position adjustment on the display device 118.

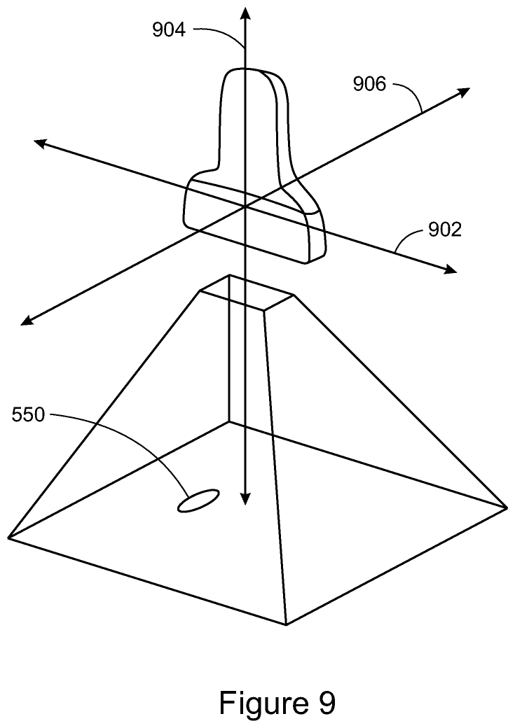

FIG. 9 is a representation of an ultrasound probe with respect to three axes and a scanned volume in accordance with an exemplary embodiment. FIG. 9 includes three axes with respect to the ultrasound probe 106. FIG. 9 includes an x-axis 902, a y-axis 904, and a z-axis 906. The x-axis 902 corresponds with an azimuth direction, the y-axis 904 corresponds with a depth direction, and the z-axis corresponds with an elevation direction.

According to an embodiment, the probe position adjustment may include one or more of a pitch adjustment, a yaw adjustment, or a roll adjustment. With respect to FIG. 9, the pitch adjustment is a rotation of the ultrasound probe 106 about the x-axis 902, the roll adjustment is a rotation of the ultrasound probe 106 about the z-axis 906, and the yaw adjustment is a rotation of the ultrasound probe about the y-axis 904. According to other embodiments, the probe position adjustment may include a translation in any direction. The probe position adjustment may include one or more of a pitch adjustment, a yaw adjustment, a roll adjustment, or a translation according to various embodiments.

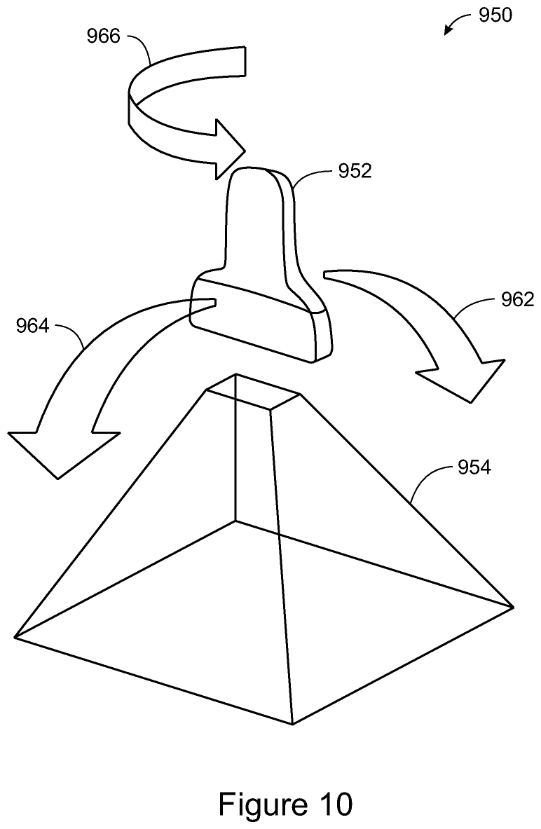

The probe position adjustment may be presented to the user using one or more graphical icons displayed on the display device 118. FIG. 10 is a representation of a graphical display in accordance with an exemplary embodiment. FIG. 10 is an example of a graphical display 950 that may be used to illustrate the probe position adjustment according to an exemplary embodiment. The graphical display 950 includes an ultrasound probe icon 952 representing the ultrasound probe 106, a schematic representation of the scanned volume 954, a first arrow 962, a second arrow 964, and a third arrow 966. The first arrow 962, the second arrow 964, and the third arrow 966 are used to represent the probe position adjustment that needs to be applied to the ultrasound probe 106 in order to position the ultrasound probe 106 in the desired position and orientation. According to an exemplary embodiment, the first arrow 962 is used to indicate a roll adjustment that should be applied to the ultrasound probe 106; the second arrow 964 is used to indicate a pitch adjustment that should be applied to the ultrasound probe 106; and the third arrow 966 is used to indicate a yaw adjustment that should be applied to the ultrasound probe 106. The first arrow 962, the second arrow 964, and the third arrow 966 each graphically illustrate the direction of the desired probe position adjustment with respect to the ultrasound probe 106 as represented by the ultrasound probe icon 952. While the probe position adjustment illustrated in FIG. 10 includes a pitch adjustment, a yaw adjustment, and a roll adjustment, it should be appreciated that the probe position adjustment in other embodiments may include an arrow indicating a desired translation. For example, the arrow may indicate the desired translation direction of the ultrasound probe. Additionally, other embodiments may display a different number of arrows to indicate the probe position adjustment. For example, some probe position adjustments may be graphically represented on the display device 118 with only a single arrow, some probe position adjustments may be graphically represented on the display device 118 with two arrows, and some probe position adjustments may be graphically represented with more than three arrows. Additionally, various embodiments may use icons other than arrows to illustrate the desired probe position adjustment during step 214.

According to an exemplary embodiment, displaying the probe position adjustment may include displaying one or more text strings for adjusting the ultrasound probe 106. For example, the processor 116 may be configured to display one or more text strings, such as, “rotate probe clockwise 30 degrees”, “tilt probe 20 degrees towards the patient's head.” “translate probe away from centerline of the patient”, etc. on the display device 118. According to other embodiments, the text strings may be presented with respect to the x-axis 902, the y-axis 904, and/or the z-axis 906. The text strings may also be presented according to any other standard reference directions such as a pitch adjustment, a yaw adjustment, and/or a roll adjustment; or a tilt adjustment, a rocking adjustment, and/or a rotation adjustment. Those skilled in the art should appreciate that the processor 116 may be configured to display any other text strings in order to communicate the desired probe position adjustment to the user.

According to other embodiments, the processor 116 may be configured to graphically display the probe position adjustment using a video sequence or a video loop. For example, the processor 116 may be configured to display a video sequence or a video loop including two or more frames showing how the ultrasound probe 106 needs to be adjusted from the current probe position to the desired probe position.

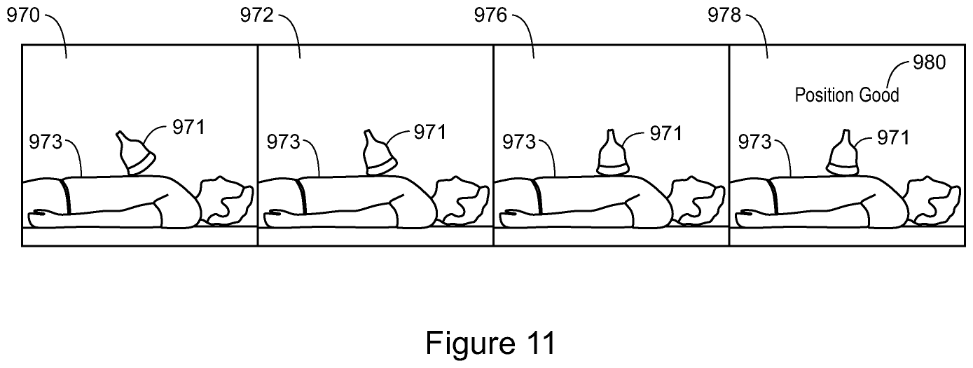

FIG. 11 is a representation of four frames of a video loop that may be displayed in a sequence or repeating loop to convey the probe position adjustment in accordance with an exemplary embodiment. FIG. 11 includes a first frame 970, a second frame 972, a third frame 976, and a fourth frame 978. In each of the frames, there is a probe icon 971 and a model of the patient 973. The position of the probe icon 971 with respect to the model of the patient 973 is different in each of the video frames. When the frames are displayed in sequence or as part of a video loop, the user can easily see how to adjust the position of the ultrasound probe 106 based on how the position of the probe icon 971 is moved as the video loop is displayed on the display device. In the example shown in FIG. 11, the fourth frame 978 includes a text string 980 stating, “Position Good”. The text string 980 indicates that the position of the probe icon 971 with respect to the model of the patient 973 is the desired position of the probe. The video loop may include a different number of frames than the four frames represented in FIG. 11 according to various embodiments. Additionally, the video loop may be configured to play at a relatively high frame rate, such as greater than 10 frames per second to show the motion of the probe icon 971 smoothly, or the video loop may be configured to play slower, such as less than 10 frames per second, which results in choppier motion between frames. The frames of the video loop may include a graphical representation of one or more scan planes (not shown) with respect to the probe icon 971 in order to help the clinician more easily understand the desired probe position adjustment.

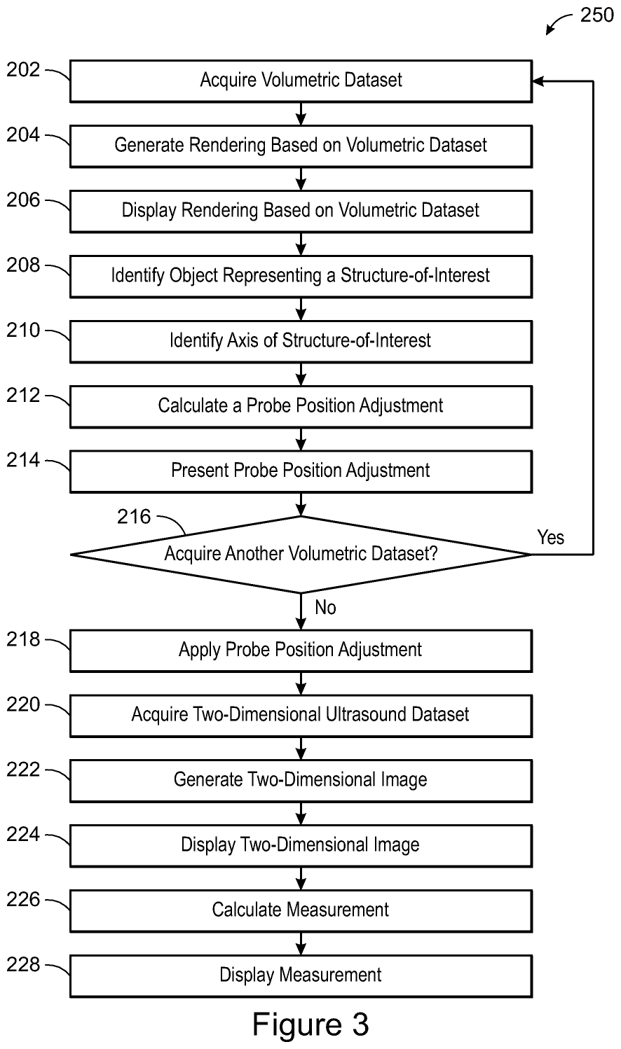

FIG. 3 is a flow chart of a method 250 in accordance with an exemplary embodiment. The individual blocks of the flow chart represent steps that may be performed in accordance with the method 250. Additional embodiments may perform the steps shown in a different sequence and/or additional embodiments may include additional steps not shown in FIG. 3. The technical effect of the method 250 is the calculation and display of a probe position adjustment with respect to a current probe position of the ultrasound probe 106. FIG. 3 provides the additional technical effect of displaying a measurement calculated from the two-dimensional image. The method 250 will be described according to an embodiment where it is performed with the ultrasound imaging system 100 shown in FIG. 1. Steps 202, 204, 206, 208, 210, 212, 214, and 216 shown in the method 250 are identical to steps 202, 204, 206, 208, 210, 212, 214, and 216 that were previously described with respect to the method 200 and will therefore not be descried again with respect to the method 250. It should be appreciated by those skilled in the art that the method 200 may be performed with other ultrasound imaging systems according to various embodiments. The method 250 will be described in detail hereinafter.

At step 216, the processor 116 determines if it is desired to acquire another volumetric dataset. If it is desired to acquire another volumetric dataset, the method 250 returns from step 216 to step 202. Steps 202, 204, 206, 208, 210, 212, 214, and 216 may be iteratively performed each time it is desired to acquire another volumetric dataset at step 216. If it is not desired to acquire another volumetric dataset at step 216, the method 250 advances to step 218.

At step 218, the clinician applies the probe position adjustment calculated at step 212 to the ultrasound probe 106. Those skilled in the art should appreciate that the probe position adjustment is applied to the ultrasound probe 106 from the current probe position. Next, at step 220, after the probe position adjustment has been applied to the ultrasound probe 106, the processor 116 controls the ultrasound probe 106 to acquire a two-dimensional ultrasound dataset of the target scan plane. As discussed hereinabove, the target scan plane is selected so that it either includes and is either parallel to an axis of the structure-of-interest or is perpendicular to an axis of the structure-of-interest. Next, at step 222, the processor 116 generates a two-dimensional image based on the two-dimensional ultrasound dataset acquired as step 220. At step 224, the processor 116 displays the two-dimensional image on the display device 118.

While not shown in FIG. 3, according to other embodiments, the processor 116 may be configured to control the ultrasound probe to acquire an updated volumetric dataset after the probe position adjustment has been applied to the ultrasound probe 106. The processor 116 may be further configured to generate at least one rendering based on the updated volumetric dataset and display the at least one rendering on the display device 118. The user may, for instance, view this at least one rendering prior to switching to the two-dimensional acquisition mode. The rendering may, for instance, be used to confirm that the probe position is correct prior to switching to the two-dimensional acquisition mode. According to an embodiment, the at least one rendering may be a A-plane of the target scan plane.

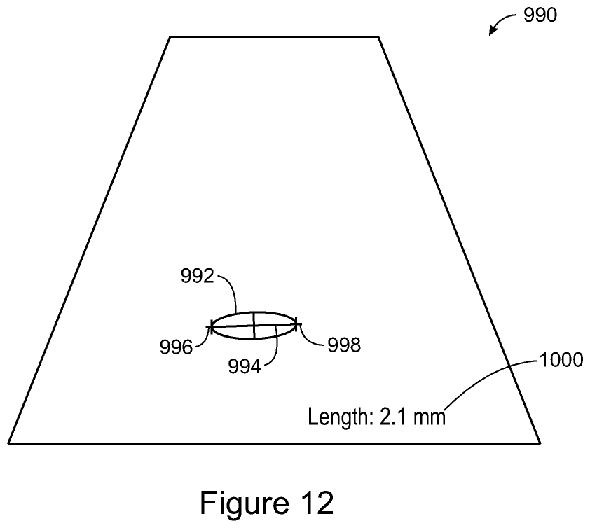

FIG. 12 is a representation of a two-dimensional image 990 in accordance with an exemplary embodiment. The two-dimensional image 990 is generated based on the two-dimensional ultrasound dataset acquired at step 220 according to an embodiment. In the two-dimensional image 990, an object 992 representing the structure-of-interest 550 is clearly represented. A line 994 is a representation of the long axis in the two-dimensional image 990. The line 994 representing the long-axis 560 is clearly visible on the two-dimensional image 990 because the two-dimensional ultrasound dataset was acquired from the target scan plane including the long-axis 560 according to an exemplary embodiment.

The two-dimensional image 990 is generated from a two-dimensional ultrasound dataset acquired of the target scan plane. The two-dimensional image 990 is not generated based on a multi-planar reformat of volumetric ultrasound dataset. Since the two-dimensional image 990 is generated from a two-dimensional ultrasound dataset, the image quality and the image resolution are much higher quality compared to a multi-planar reformat based on a volumetric ultrasound dataset. Furthermore, in the embodiment shown in FIG. 12, the long axis is included in the target scan plane. This means that the two-dimensional image 990 is well-suited for performing any measurements related to the long axis.

According to an exemplary embodiment shown in FIG. 3, the method 250 advances to step 226, where the processor 116 calculates a measurement based on the two-dimensional image 990. According to an exemplary embodiment, the processor 116 may be configured to calculate a length of the long axis. The processor 116 may, for instance, be configured to identify a first end point 996 of the line 994 and a second end point 998 of the line 994. As discussed previously, the line 996 corresponds with the long-axis 560 of the structure-of-interest 550. The processor 116 may be configured to identify the first end point 996 and the second end point 998 by identifying the respective locations on the two-dimensional image 990 where the line 994 intersects a boundary of the object 992. Once the first end point 996 and the second end point 994 have been identified, the processor 116 may be configured to calculate the straight-line length of the line 992. This length represents the length of the long-axis 560 according to an embodiment. Next, at step 228, the processor 116 displays the measurement on the display device 118. For example, the two-dimensional image 990 includes a text string 1000 that says, “Length: 2.1 mm.” According to an embodiment, 2.1 mm is the length of the long-axis 560 of the structure-of-interest 550 as determined based on the object 992 shown in the two-dimensional image 990.

According to other embodiments, the user may manually identify two or more points on the two-dimensional image that are used in the calculation of the measurement. This type of measurement may be referred to as implementing a “calipers” measurement technique. For example, the user may use one or more controls that are part of the user interface 115 to position points, such as the first end point 996 and the second end point 998, on the two-dimensional image 990. The user may, for instance, use a trackball, a touchpad, a touchscreen, a mouse, etc. to identify the positions of each point on the two-dimensional image.

According to other embodiments, points on the two-dimensional image may be identified using a semi-automated process. For example, the processor 116 may display a suggested location for each point and the user may be able to either accept each point or adjust the position of one or more of the suggested locations the points. For example, the user may use one or more user input devices that are part of the user interface 115 to adjust the position of each location suggested by the processor 116 if desired. The user may, for instance, use a trackball, a touchpad, a touchscreen, a mouse, etc. to reposition each point from a suggested location if the user is not satisfied with the suggested location provided by the processor 116.

According to other embodiments, the processor 116 may be configured to calculate different measurements based on the displayed two-dimensional image. For example, the processor 116 may be configured to calculate any other measurements including an area, a circumference, a diameter, etc. based on the two-dimensional image. These other measurements may use the placement of two or more points, as was described with respect to the length measurement, or they may involve the placement of a line, curve, contour, etc. based on the information in the two-dimensional image. The processor 116 may be configured to use image processing techniques, such as thresholding, to determine where to place the line, the curve, the contour, etc. that will be used to calculate the measurement on the two-dimensional image 990. It should be appreciated by those skilled in the art that the processor 116 may be configured to calculate the measurement using different techniques according to various embodiments, and/or the processor 116 may be configured to calculate other measurements than the one explicitly described hereinabove according to various embodiments.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

We claim:

1. A method of ultrasound imaging, the method comprising:
acquiring a volumetric dataset with an ultrasound probe in a volumetric acquisition mode;
automatically identifying, with a processor, an object representing a structure-of-interest from the volumetric dataset;
automatically identifying, with the processor, an axis of the structure-of-interest based on the object;
automatically calculating, with the processor, a probe position adjustment from a current probe position to enable the acquisition of a target scan plane of the structure-of-interest that either includes and is parallel to the axis or is perpendicular to the axis; and
presenting the probe position adjustment on a display device.

2. The method of claim 1, further comprising:
applying the probe position adjustment to the ultrasound probe from the current probe position;
acquiring a two-dimensional ultrasound dataset of the target scan plane with the ultrasound probe in a two-dimensional acquisition mode after applying the probe position adjustment;
generating a two-dimensional image based on the two-dimensional ultrasound dataset; and
displaying the two-dimensional image on the display device.

3. The method of claim 2, further comprising:
calculating a measurement of the structure-of-interest along the axis based on the representation of the axis in the two-dimensional image; and
displaying the measurement on the display device.

4. The method of claim 1, wherein the probe position adjustment comprises one or more of a pitch adjustment, a yaw adjustment, or a roll adjustment.

5. The method of claim 1, wherein the probe position adjustment to the ultrasound probe position comprises a translation adjustment and one or more of a pitch adjustment, a yaw adjustment, or a roll adjustment.

6. The method of claim 1, wherein said automatically identifying the object from the volumetric dataset comprises implementing an artificial intelligence technique with the processor.

7. The method of claim 6, wherein the artificial intelligence technique is a neural network.

8. The method of claim 1, wherein said automatically identifying the axis comprises implementing, with the processor, an artificial intelligence technique.

9. The method of claim 1, wherein said automatically identifying the object from the volumetric dataset comprises implementing a first artificial intelligence technique with the processor, and wherein said automatically identifying the axis comprises implementing a second artificial intelligence technique with the processor.

10. The method of claim 9, wherein the first artificial intelligence technique is a U-Net network, and the second artificial intelligence technique is convolutional neural network.

11. An ultrasound imaging system comprising:
an ultrasound probe;
a display device; and
a processor in electronic communication with both the ultrasound probe and the display device, wherein the processor is configured to:
control the ultrasound probe to acquire a volumetric dataset in a volumetric acquisition mode;
automatically identify an object from the volumetric dataset representing a structure-of-interest;
automatically identify an axis of the structure-of-interest based on the object;
automatically calculate a probe position adjustment from a current probe position to enable the acquisition of a target scan plane of the structure-of-interest that either includes and is parallel to the axis or is perpendicular to the axis; and
present the probe position adjustment on the display device.

12. The ultrasound imaging system of claim 11, wherein the processor is further configured to:
control the ultrasound probe to acquire a two-dimensional dataset of the target scan plane in a two-dimensional acquisition mode after the probe position adjustment has been applied to the ultrasound probe;
generate a two-dimensional image based on the two-dimensional ultrasound dataset; and
display the two-dimensional image on the display device.

13. The ultrasound imaging system of claim 11, wherein the processor is further configured to:
calculate a measurement of the structure-of-interest along the axis based on the representation of the axis in the two-dimensional image; and
display the measurement on the display device.

14. The ultrasound imaging system of claim 11, wherein the probe position adjustment presented on the display device comprises one or more of a pitch adjustment, a yaw adjustment, or a roll adjustment.

15. The ultrasound imaging system of claim 11, wherein the processor is configured to present the probe position adjustment by displaying one or more arrows in relation to an ultrasound probe icon displayed on the display device.

16. The ultrasound imaging system of claim 11, wherein the processor is configured to implement an artificial intelligence technique to identify the object.

17. The ultrasound imaging system of claim 16, wherein the artificial intelligence technique is a neural network.

18. The ultrasound imaging system of claim 11, wherein the processor is configured to implement an artificial intelligence technique to identify the axis.

19. The ultrasound imaging system of claim 11, where the processor is further configured to:
control the ultrasound probe to acquire an updated volumetric dataset after the probe position adjustment has been applied to the ultrasound probe;
generate at least one rendering based on the updated volumetric dataset; and
display the at least one rendering on the display device.

20. The ultrasound imaging system of claim 19, wherein the at least one rendering comprises an A-plane of the target scan plane.
